(19) United States
(12) Patent Application Publication
(10) Pub. No.: US 2009/0053724 A1
(43) Pub. Date: Feb. 26, 2009

(54) System and method for adaptive reagent control in nucleic acid sequencing

(75) Inventors: George Thomas Roth, Fairfield, CT (US); John Richard Nobile, Fairfield, CT (US); Maithreyan Srinivasan, Mountain View, CA (US); Zhoutao Chen, Milford, CT (US); James Matthew Nealis, East Lyme, CT (US); Xavier Victor Gomes, Wallingford, CT (US)

Correspondence Address: Ivor R. Elrifi;Mintz, Levin, Cohn, Ferris, Glovsky and Popeo, P.C, 666 Third Avenue - 24th Floor, New York, NY, 10017, US

(73) Assignee: 454 Life Sciences Corporation

(21) Appl. No.: 12/215,455

(22) Filed: Jun. 27, 2008

Related U.S. Application Data
(60) Provisional application No. 60/946,743, filed on Jun. 28, 2007.

(51) Int. Cl. C12Q 1/68; C12Q 1/00; C12M 1/36; C12M 1/34; C12Q 1/34

(52) U.S. Cl. 435/6; 435/4; 435/18; 435/286.5

(57) ABSTRACT

An embodiment of a method for adaptive reagent control is described that comprising a) introducing a first concentration of an enzyme reagent into a reaction environment with a reaction substrate, where the enzyme reagent and reaction substrate are constituent parts of a sequencing process; b) measuring a level of activity of the first concentration of the enzyme reagent in the reaction environment, where the level of activity comprises a measurable product of a reaction between the enzyme reagent and the reaction substrate; c) identifying an optimal concentration using the measured level of activity of the first concentration; and d) performing the sequencing process in the reaction environment using the optimal concentration of the enzyme reagent, where the sequencing process comprises an iterative series of sequencing reactions.

### RELATED APPLICATIONS

The present application is related to and claims priority from U.S. Provisional Patent Application Ser. No. 60/946,743, titled “System and Method for Adaptive Reagent Control in Nucleic Acid Sequencing”, filed Jun. 28, 2007, which is hereby incorporated by reference herein in its entirety for all purposes.

### FIELD OF THE INVENTION

The present invention relates to the fields of molecular biology and one or more adaptive reagent control methods and elements. More specifically, the invention relates to measuring the activity of and dynamically adjusting the concentration of one or more enzyme reagents employed in nucleic acid sequencing processes to optimize the performance and increase the efficiency of said reagents and processes. Further, the invention relates to instrumentation that enables automated measurement and modulation of reagent concentration.

### BACKGROUND OF THE INVENTION

There are a number of “sequencing” techniques known in the art amenable for use with the presently described invention such as, for instance, techniques based upon what are referred to as Sanger sequencing methods commonly known to those of ordinary skill in the art that employ termination and size separation techniques. Another class of powerful sequencing techniques includes what are referred to as “Sequencing-by-synthesis” techniques (SBS). SBS techniques are generally employed for determining the identity or nucleic acid composition of one or more molecules in a nucleic acid sample. SBS techniques provide many desirable advantages over previously employed sequencing techniques. For example, embodiments of SBS are enabled to perform what are referred to as high throughput sequencing that generates a large volume of high quality sequence information at a low cost relative to previous techniques. A further advantage includes the simultaneous generation of sequence information from multiple template molecules in a massively parallel fashion. In other words, multiple nucleic acid molecules derived from one or more samples are simultaneously sequenced in a single process.

Typical embodiments of SBS methods comprise the stepwise synthesis of a single strand of polynucleotide molecule complementary to a template nucleic acid molecule whose nucleotide sequence composition is to be determined. For example, SBS techniques typically operate by adding a single nucleic acid (also referred to as a nucleotide) species to a nascent polynucleotide molecule complementary to a nucleic acid species of a template molecule at a corresponding sequence position. The addition of the nucleic acid species to the nascent molecule is generally detected using a variety of methods known in the art that include, but are not limited to what are referred to as pyrosequencing or fluorescent detection methods such as those that employ reversible terminators or energy transfer labels including fluorescent resonant energy transfer dyes (FRET). Typically, the process is iterative until a complete (i.e. all sequence positions are represented) or desired sequence length complementary to the template is synthesized.

Further, as described above many embodiments of SBS are enabled to perform sequencing operations in a massively parallel manner. For example, some embodiments of SBS methods are performed using instrumentation that automates one or more steps or operation associated with the preparation and/or sequencing methods. Some instruments employ elements such as plates with wells or other type of microreactor configuration that provide the ability to perform reactions in each of the wells or microreactors simultaneously. Additional examples of SBS techniques as well as systems and methods for massively parallel sequencing are described in U.S. Pat. Nos. 6,274,320; 6,258,568; 6,210,891, 7,211,390; 7,244,559; 7,264,929; 7,335,762; and 7,323,305 each of which is hereby incorporated by reference herein in its entirety for all purposes; and U.S. patent application Ser. No. 11/195,254, which is hereby incorporated by reference herein in its entirety for all purposes.

It will be appreciated that typical embodiments of SBS are sensitive to differences in parameters associated with various elements employed in process steps or components such as, for instance, varying levels of catalytic activity associated with enzymatic process steps. Therefore, it is generally desirable in embodiments of SBS to employ strategies or methods that improve the efficiency of one or more process steps or components. For example, it is generally appreciated that all molecules of a particular nucleotide specie employed in a previous extension cycle should be removed and/or inactivated prior to the initiation of the subsequent extension reaction with a different nucleotide specie in the next cycle. If remnants of a nucleotide specie from a previous cycle remain in the current cycle of a different nucleotide specie it is likely that some of the remnant nucleotide specie molecules will be incorporated into the nascent molecule. The incorporation of the remnant nucleotide specie molecules would be erroneously interpreted as an incorporation of the nucleotide specie of the present cycle. In the present example, incorporation of unintended nucleotide specie molecules may promote what are referred to as carry forward effects which are described in greater detail below. It is therefore advantageous to employ one or more methods to ensure the complete removal or inactivation of leftover nucleotide specie molecules as well as other undesirable reaction products or reagents.

One method that is particularly efficient and amenable for use with SBS methods is to wash a reaction vessel or substrate area with what is referred to as “apyrase”. Those of ordinary skill in the related art will appreciate that apyrase is an enzyme that has a number of qualities that include the degradation of nucleoside triphosphates, diphosphates, ATP, and PPi (pyrophosphate). The use of apyrase in SBS embodiments substantially improves the removal of excess and unwanted nucleotide species, reagents, and reaction products over simply washing alone. For example, apyrase may be “washed” or “flowed” over a surface of a solid support comprising one or more reaction areas at the end of each reaction cycle so as to facilitate the degradation of any remaining, non-incorporated nucleotide specie molecules within the sequencing reaction mixture. Apyrase may further be employed to degrade ATP generated in a previous cycle and hence “turns off” light generated from the reaction in the previous cycle.

The next reaction cycle with a different nucleotide specie may be initiated after a brief washing step that removes remaining apyrase and reaction products. In some embodiments, the apyrase may be bound to the solid or mobile solid support. Additional examples of apyrase use and the advantages conferred by such use are described in U.S. Pat. No. 7,323,305, titled “Methods of amplifying and sequencing nucleic acids”, which is hereby incorporated by reference herein in its entirety for all purposes.

In typical embodiments, it is critically important to employ the correct concentration of apyrase to avoid undesirable effects. For example, if the concentration of apyrase is too high the result may include the degradation of the desired nucleotide species in a subsequent cycle. In other words, unreacted apyrase may still be present at the beginning of a reaction cycle due to the high concentration which in turn degrades the nucleotide specie molecules introduced in that flow. Such an excess of apyrase activity promotes what is referred to as “incomplete extension” effects. Alternatively, if the apyrase concentration or activity is too low the result may include some portion or percentage of the nucleotide species from a previous cycle present in a current cycle. As described above, low or absent apyrase activity promotes what is referred to as “carry forward” effects. In the present example, it is therefore generally desirable to measure apyrase activity so that the concentration may be modulated to provide an optimal level of activity in a reaction.

As described above, carry forward and incomplete extension effects may be the result of non-optimal apyrase concentration or activity and are two important sources of error to consider. For example, a small fraction of template nucleic acid molecules in each amplified population from a sample (i.e. a population of substantially identical copies amplified from a nucleic acid molecule template) loses or falls out of phasic synchronism with the rest of the template nucleic acid molecules in the population (that is, the reactions associated with the fraction of template molecules either get ahead of, or fall behind, the interrogated sequence position of the other template molecules in the sequencing reaction run on the population). In the present example, the failure of the reaction to properly incorporate one or more nucleotide species into one or more nascent molecules for extension of the sequence by one position results in each subsequent reaction being at a sequence position that is behind and out of phase with the sequence position of the rest of the population. This effect is referred to herein as “incomplete extension” (IE). Alternatively, the improper extension of a nascent molecule by incorporation of one or more nucleotide species in a sequence position that is ahead and out of phase with the sequence position of the rest of the population is referred to herein as “carry forward” (CF). The combined effects of CF and IE are referred to herein as CAFIE. Phasic synchrony error and methods of correction are further described in PCT Application Ser. No. US2007/004187, titled “System and Method for Correcting Primer Extension Errors in Nucleic Acid Sequence Data”, filed Feb. 15, 2007 which is hereby incorporated by reference herein in its entirety for all purposes.

Therefore, it is significantly advantageous to employ methods to measure and modulate the concentration of apyrase as well as other important reagents in SBS methods and processes. It is particularly useful in automated embodiments performed using various instruments where it is further desirable to be able to dynamically measure the concentration or effectiveness of an enzyme or reagent where the concentration of said enzymes or reagents may be adaptively modified to match the needs of the system with the measurements.

### SUMMARY OF THE INVENTION

Embodiments of the invention relate to the determination of the sequence of nucleic acids. More particularly, embodiments of the invention relate to methods and systems for correcting errors in data obtained during the sequencing of nucleic acids by SBS.

An embodiment of a method for adaptive reagent control is described that comprising a) introducing a first concentration of an enzyme reagent into a reaction environment with a reaction substrate, where the enzyme reagent and reaction substrate are constituent parts of a sequencing process; b) measuring a level of activity of the first concentration of the enzyme reagent in the reaction environment, where the level of activity comprises a measurable product of a reaction between the enzyme reagent and the reaction substrate; c) identifying an optimal concentration using the measured level of activity of the first concentration; and d) performing the sequencing process in the reaction environment using the optimal concentration of the enzyme reagent, where the sequencing process comprises an iterative series of sequencing reactions.

In some implementations the method also comprises before step d), repeating steps a) and b) using the optimal concentration as the first concentration; and verifying the optimal concentration of the enzyme reagent using the measured level of activity. In addition the method may also comprise introducing a second concentration and a third concentration of the enzyme reagent into the reaction environment with the reaction substrate; measuring a level of activity of the second and third concentrations of the enzyme reagent in the reaction environment; and identifying the optimal concentration using the measured level of activity of the first, second and third concentrations.

An embodiment of a nucleic acid sequencing system is also described that comprises a flow cell that includes a reaction environment for performing a sequencing process comprising an iterative series of sequencing reactions; a valve that introduces a first concentration of an enzyme reagent into a reaction environment with a reaction substrate, wherein the enzyme reagent and reaction substrate are constituent parts of the sequencing process; a detector that measures a level of activity of the first concentration of the enzyme reagent in the reaction environment, wherein the level of activity comprises a measurable product of a reaction between the enzyme reagent and the reaction substrate; where the valve provides an optimal concentration of the enzyme reagent into the reaction environment in response to the measured level of activity.

In some implementations, the system further comprises a computer having executable code stored thereon, where the executable code performs the steps of: providing instructional control for the valve to introduce the first concentration of the enzyme reagent and the reaction substrate into the reaction environment; receiving the measured level of activity of the first concentration from the detector; identifying an optimal concentration using the measured level of activity of the first concentration; and providing instructional control for the valve to provide the optimal concentration.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 160 appears first in FIG. 1). All of these conventions, however, are intended to be typical or illustrative, rather than limiting.

FIG. 1 is a functional block diagram of one embodiment of a sequencing instrument comprising optic and fluidic subsystems for processing a reaction substrate under computer control;

FIG. 2 is a functional block diagram of one embodiment of the optic and fluidic subsystems of FIG. 1 for processing the reaction substrate;

FIG. 3 is a simplified graphical representation of measured variation in the activity levels of multiple samples of an enzyme reagent;

FIGS. 4A and 4B are simplified graphical representations of one embodiment of a test of enzymatic activity using a test molecule illustrating a difference between a sequencing run without error and a sequencing run with introduced error; and

FIG. 5 is a simplified graphical representation of one embodiment of the relationship between measured signals and concentration of an enzyme reagent.

### DETAILED DESCRIPTION OF THE INVENTION

As will be described in greater detail below, embodiments of the presently described invention include systems and methods for adaptive control of reagent concentration employed in sequencing reactions. Further, the invention includes dynamically measuring the concentration or activity of the reagents prior to and/or during the sequencing process and modulating the concentration so that the reagent activity is within an optimal range for the sequencing process.

a. General

The terms “flowgram” and “pyrogram” may be used interchangeably herein and generally refer to a graphical representation of sequence data generated by SBS methods.

Further, the term “read” or “sequence read” as used herein generally refers to the entire sequence data obtained from a single nucleic acid template molecule or a population of a plurality of substantially identical copies of the template nucleic acid molecule.

The terms “run” or “sequencing run” as used herein generally refer to a series of sequencing reactions performed in a sequencing operation of one or more template nucleic acid molecule.

The term “flow” as used herein generally refers to a serial or iterative cycle of addition of solution to an environment comprising a template nucleic acid molecule, where the solution may include a nucleotide specie for addition to a nascent molecule or other reagent such as buffers or enzymes that may be employed to reduce carryover or noise effects from previous flow cycles of nucleotide specie.

The term “flow cycle” as used herein generally refers to a sequential series of flows where a nucleotide species is flowed once during the cycle (i.e. a flow cycle may include a sequential addition in the order of T, A, C, G nucleotide species, although other sequence combinations are also considered part of the definition). Typically the flow cycle is a repeating cycle having the same sequence of flows from cycle to cycle.

The term “read length” as used herein generally refers to an upper limit of the length of a template molecule that may be reliably sequenced. There are numerous factors that contribute to the read length of a system and/or process including, but not limited to the degree of GC content in a template nucleic acid molecule.

The term “test fragment”, or “TF” as used herein generally refers to a nucleic acid element of known sequence composition that may be employed for quality control, calibration, or other related purposes.

A “nascent molecule” generally refers to a DNA strand which is being extended by the template-dependent DNA polymerase by incorporation of nucleotide species which are complementary to the corresponding nucleotide species in the template molecule.

The terms “template nucleic acid”, “template molecule”, “target nucleic acid”, or “target molecule” generally refer to a nucleic acid molecule that is the subject of a sequencing reaction from which sequence data or information is generated.

The term “nucleotide specie” as used herein generally refers to the identity of a nucleic acid monomer including purines (Adenine, Guanine) and pyrimidines (Cytosine, Uracil, Thymine) typically incorporated into a nascent nucleic acid molecule.

The term “monomer repeat” or “homopolymers” as used herein generally refers to two or more sequence positions comprising the same nucleotide specie (i.e. a repeated nucleotide specie).

The term “homogeneous extension”, as used herein, generally refers to the relationship or phase of an extension reaction where each member of a population of substantially identical template molecules is homogenously performing the same extension step in the reaction.

The term “completion efficiency” as used herein generally refers to the percentage of nascent molecules that are properly extended during a given flow.

The term “incomplete extension rate” as used herein generally refers to the ratio of the number of nascent molecules that fail to be properly extended over the number of all nascent molecules.

The term “genomic library” or “shotgun library” as used herein generally refers to a collection of molecules derived from and/or representing an entire genome (i.e. all regions of a genome) of an organism or individual.

The term “amplicon” as used herein generally refers to selected amplification products such as those produced from Polymerase Chain Reaction or Ligase Chain Reaction techniques.

The term “keypass” or “keypass mapping” as used herein generally refers to a nucleic acid “key element” associated with a template nucleic acid molecule in a known location (i.e. typically included in a ligated adaptor element) comprising known sequence composition that is employed as a quality control reference for sequence data generated from template molecules. The sequence data passes the quality control if it includes the known sequence composition associated with a Key element in the correct location.

The term “blunt end” or “blunt ended” as used herein generally refers to a linear double stranded nucleic acid molecule having an end that terminates with a pair of complementary nucleotide base species, where a pair of blunt ends are always compatible for ligation to each other.

Some exemplary embodiments of systems and methods associated with sample preparation and processing, generation of sequence data, and analysis of sequence data are generally described below, some or all of which are amenable for use with embodiments of the presently described invention. In particular the exemplary embodiments of systems and methods for preparation of template nucleic acid molecules, amplification of template molecules, generating target specific amplicons and/or genomic libraries, sequencing methods and instrumentation, and computer systems are described.

In typical embodiments, the nucleic acid molecules derived from an experimental or diagnostic sample must be prepared and processed from its raw form into template molecules amenable for high throughput sequencing. The processing methods may vary from application to application resulting in template molecules comprising various characteristics. For example, in some embodiments of high throughput sequencing it is preferable to generate template molecules with a sequence or read length that is at least the length a particular sequencing method can accurately produce sequence data for. In the present example, the length may include a range of about 25-30 base pairs, about 50-100 base pairs, about 200-300 base pairs, or about 350-500 base pairs, or other length amenable for a particular sequencing application. In some embodiments, nucleic acids from a sample, such as a genomic sample, are fragmented using a number of methods known to those of ordinary skill in the art. In preferred embodiments, methods that randomly fragment (i.e. do not select for specific sequences or regions) nucleic acids and may include what is referred to as nebulization or sonication methods. It will however, be appreciated that other methods of fragmentation such as digestion using restriction endonucleases may be employed for fragmentation purposes. Also in the present example, some processing methods may employ size selection methods known in the art to selectively isolate nucleic acid fragments of the desired length.

Also, it is preferable in some embodiments to associate additional functional elements with each template nucleic acid molecule. The elements may be employed for a variety of functions including, but not limited to, primer sequences for amplification and/or sequencing methods, quality control elements, unique identifiers that encode various associations such as with a sample of origin or patient, or other functional element. For example, some embodiments may associate priming sequence elements or regions comprising complementary sequence composition to primer sequences employed for amplification and/or sequencing. Further, the same elements may be employed for what may be referred to as “strand selection” and immobilization of nucleic acid molecules to a solid phase substrate. In the present example, two sets of priming sequence regions (hereafter referred to as priming sequence A, and priming sequence B) may be employed for strand selection where only single strands having one copy of priming sequence A and one copy of priming sequence B is selected and included as the prepared sample. The same priming sequence regions may be employed in methods for amplification and immobilization where, for instance priming sequence B may be immobilized upon a solid substrate and amplified products are extended therefrom.

Additional examples of sample processing for fragmentation, strand selection, and addition of functional elements and adaptors are described in U.S. patent application Ser. No. 10/767,894, titled “Method for preparing single-stranded DNA libraries”, filed Jan. 28, 2004; U.S. Provisional Patent Application Ser. No. 61/031,779, titled “System and Method for Improved Processing of Nucleic Acids for Production of Sequencable Libraries”, filed Feb. 27, 2008; and Attorney Docket No. 21465-529001 US, titled “System and Method for Identification of Individual Samples from a Multiplex Mixture”, filed May 29, 2008, each of which is hereby incorporated by reference herein in its entirety for all purposes.

Various examples of systems and methods for performing amplification of template nucleic acid molecules to generate populations of substantially identical copies are described. It will be apparent to those of ordinary skill that it is desirable in some embodiments of SBS to generate many copies of each nucleic acid element to generate a stronger signal when one or more nucleotide species is incorporated into each nascent molecule associated with a copy of the template molecule. There are many techniques known in the art for generating copies of nucleic acid molecules such as, for instance, amplification using what are referred to as bacterial vectors, “Rolling Circle” amplification (described in U.S. Pat. Nos. 6,274,320 and 7,211,390, incorporated by reference above) and Polymerase Chain Reaction (PCR) methods, each of the techniques are applicable for use with the presently described invention. One PCR technique that is particularly amenable to high throughput applications include what are referred to as emulsion PCR methods (also referred to as emPCR™ methods).

Typical embodiments of emulsion PCR methods include creating a stable emulsion of two immiscible substances creating aqueous droplets within which reactions may occur. In particular, the aqueous droplets of an emulsion amenable for use in PCR methods may include a first fluid such as a water based fluid suspended or dispersed in what may be referred to as a discontinuous phase within another fluid such as an oil based fluid. Further, some emulsion embodiments may employ surfactants that act to stabilize the emulsion that may be particularly useful for specific processing methods such as PCR. Some embodiments of surfactant may include non-ionic surfactants such as sorbitan monooleate (also referred to as Span™ 80), polyoxyethylenesorbitsan monooleate (also referred to as Tween™ 80), or in some preferred embodiments dimethicone copolyol (also referred to as Abil® EM90), polysiloxane, polyalkyl polyether copolymer, polyglycerol esters, poloxamers, and PVP/hexadecane copolymers (also referred to as Unimer U-151), or in more preferred embodiments a high molecular weight silicone polyether in cyclopentasiloxane (also referred to as DC 5225C available from Dow Corning).

The droplets of an emulsion may also be referred to as compartments, microcapsules, microreactors, microenvironments, or other name commonly used in the related art. The aqueous droplets may range in size depending on the composition of the emulsion components or composition, contents contained therein, and formation technique employed. The described emulsions create the microenvironments within which chemical reactions, such as PCR, may be performed. For example, template nucleic acids and all reagents necessary to perform a desired PCR reaction may be encapsulated and chemically isolated in the droplets of an emulsion. Additional surfactants or other stabilizing agent may be employed in some embodiments to promote additional stability of the droplets as described above. Thermocycling operations typical of PCR methods may be executed using the droplets to amplify an encapsulated nucleic acid template resulting in the generation of a population comprising many substantially identical copies of the template nucleic acid. In some embodiments, the population within the droplet may be referred to as a “clonally isolated”, “compartmentalized”, “sequestered”, “encapsulated”, or “localized” population. Also in the present example, some or all of the described droplets may further encapsulate a solid substrate such as a bead for attachment of template or other type of nucleic acids, reagents, labels, or other molecules of interest.

Embodiments of an emulsion useful with the presently described invention may include a very high density of droplets or microcapsules enabling the described chemical reactions to be performed in a massively parallel way. Additional examples of emulsions employed for amplification and their uses for sequencing applications are described in U.S. patent application Ser. Nos. 10/861,930; 10/866,392; 10/767,899; 11/045,678 each of which are hereby incorporated by reference herein in its entirety for all purposes.

Also, an exemplary embodiment for generating target specific amplicons for sequencing is described that includes using sets of nucleic acid primers to amplify a selected target region or regions from a sample comprising the target nucleic acid. Further, the sample may include a population of nucleic acid molecules that are known or suspected to contain sequence variants and the primers may be employed to amplify and provide insight into the distribution of sequence variants in the sample.

For example a method for identifying a sequence variant by specific amplification and sequencing of multiple alleles in a nucleic acid sample may be performed. The nucleic acid is first subjected to amplification by a pair of PCR primers designed to amplify a region surrounding the region of interest or segment common to the nucleic acid population. Each of the products of the PCR reaction (amplicons) is subsequently further amplified individually in separate reaction vessels such as an emulsion based vessel described above. The resulting amplicons (referred to herein as second amplicons), each derived from one member of the first population of amplicons, are sequenced and the collection of sequences, from different emulsion PCR amplicons, are used to determine an allelic frequency.

Some advantages of the described target specific amplification and sequencing methods include a higher level of sensitivity than previously achieved. Further, embodiments that employ high throughput sequencing instrumentation such as for instance embodiments that employ what is referred to as a PicoTiterPlate® array of wells provided by 454 Life Sciences Corporation, the described methods can be employed to sequence over 100,000 or over 300,000 different copies of an allele per run or experiment. Also, the described methods provide a sensitivity of detection of low abundance alleles which may represent 1% or less of the allelic variants. Another advantage of the methods includes generating data comprising the sequence of the analyzed region. Importantly, it is not necessary to have prior knowledge of the sequence of the locus being analyzed.

Additional examples of target specific amplicons for sequencing are described in U.S. patent application Ser. No. 11/104,781, titled “Methods for determining sequence variants using ultra-deep sequencing”, filed Apr. 12, 2005, which is hereby incorporated by reference herein in its entirety for all purposes.

Further, embodiments of sequencing may include Sanger type techniques, techniques generally referred to as Sequencing by Hybridization (SBH) or Sequencing by Incorporation (SBI) that may include what is referred to as polony sequencing techniques; nanopore, waveguide and other single molecule detection techniques; or reversible terminator techniques. As described above a preferred technique may include Sequencing by Synthesis methods. For example, some SBS embodiments sequence populations of substantially identical copies of a nucleic acid template and typically employ one or more oligonucleotide primers designed to anneal to a predetermined, complementary position of the sample template molecule or one or more adaptors attached to the template molecule. The primer/template complex is presented with a nucleotide specie in the presence of a nucleic acid polymerase enzyme. If the nucleotide specie is complementary to the nucleic acid specie corresponding to a sequence position on the sample template molecule that is directly adjacent to the 3′ end of the oligonucleotide primer, then the polymerase will extend the primer with the nucleotide specie. Alternatively, in some embodiments the primer/template complex is presented with a plurality of nucleotide species of interest (typically A, G, C, and T) at once, and the nucleotide specie that is complementary at the corresponding sequence position on the sample template molecule directly adjacent to the 3′ end of the oligonucleotide primer is incorporated. In either of the described embodiments, the nucleotide species may be chemically blocked (such as at the 3′-O position) to prevent further extension, and need to be deblocked prior to the next round of synthesis. It will also be appreciated that the process of adding a nucleotide specie to the end of a nascent molecule is substantially the same as that described above for addition to the end of a primer.

As described above, incorporation of the nucleotide specie can be detected by a variety of methods known in the art, e.g. by detecting the release of pyrophosphate (PPi) (examples described in U.S. Pat. Nos. 6,210,891; 6,258,568; and 6,828,100, each of which is hereby incorporated by reference herein in its entirety for all purposes), or via detectable labels bound to the nucleotides. Some examples of detectable labels include but are not limited to mass tags and fluorescent or chemiluminescent labels. In typical embodiments, unincorporated nucleotides are removed, for example by washing. Further, in some embodiments the unincorporated nucleotides may be subjected to enzymatic degradation such as, for instance, degradation using the apyrase enzyme as described herein. In the embodiments where detectable labels are used, they will typically have to be inactivated (e.g. by chemical cleavage or photobleaching) prior to the following cycle of synthesis. The next sequence position in the template/polymerase complex can then be queried with another nucleotide species, or a plurality of nucleotide species of interest, as described above. Repeated cycles of nucleotide addition, extension, signal acquisition, and washing result in a determination of the nucleotide sequence of the template strand. Continuing with the present example, a large number or population of substantially identical template molecules (e.g. 10³, 10⁴, 10⁵, 10⁶ or 10⁷ molecules) are typically analyzed simultaneously in any one sequencing reaction, in order to achieve a signal which is strong enough for reliable detection.

In addition, it may be advantageous in some embodiments to improve the read length capabilities and qualities of a sequencing process by employing what may be referred to as a “paired-end” sequencing strategy. For example, some embodiments of sequencing method have limitations on the total length of molecule from which a high quality and reliable read may be generated. In other words, the total number of sequence positions for a reliable read length may not exceed 25, 50, 100, or 150 bases depending on the sequencing embodiment employed. A paired-end sequencing strategy extends reliable read length by separately sequencing each end of a molecule (sometimes referred to as a “tag” end) that comprise a fragment of an original template nucleic acid molecule at each end joined in the center by a linker sequence. The original positional relationship of the template fragments is known and thus the data from the sequence reads may be re-combined into a single read having a longer high quality read length. Further examples of paired-end sequencing embodiments are described in U.S. patent application Ser. No. 11/448,462, titled “Paired end sequencing”, filed Jun. 6, 2006, and in U.S. Provisional Patent Application Ser. No. 61/026,319, titled “Paired end sequencing”, filed Feb. 5, 2008, each of which is hereby incorporated by reference herein in its entirety for all purposes.

Some examples of SBS apparatus may implement some or all of the methods described above may include one or more of a detection device such as a charge coupled device (i.e. CCD camera), a microfluidics chamber or flow cell, a reaction substrate, and/or a pump and flow valves. Taking the example of pyrophosphate based sequencing, embodiments of an apparatus may employ a chemiluminescent detection strategy that produces an inherently low level of background noise.

In some embodiments, the reaction substrate for sequencing may include what is referred to as a PicoTiterPlate® array (also referred to as a PTP® plate) formed from a fiber optics faceplate that is acid-etched to yield hundreds of thousands of very small wells each enabled to hold a population of substantially identical template molecules. In some embodiments, each population of substantially identical template molecule may be disposed upon a solid substrate such as a bead, each of which may be disposed in one of said wells. For example, an apparatus may include a reagent delivery element for providing fluid reagents to the PTP plate holders, as well as a CCD type detection device enabled to collect photons of light emitted from each well on the PTP plate. Further examples of apparatus and methods for performing SBS type sequencing and pyrophosphate sequencing are described in U.S. Pat. No 7,323,305 and U.S. patent application Ser. No. 11/195,254 both of which are incorporated by reference above.

In addition, systems and methods may be employed that automate one or more sample preparation processes, such as the emPCR™ process described above. For example, automated systems may be employed to provide an efficient solution for generating an emulsion for emPCR processing, performing PCR Thermocycling operations, and enriching for successfully prepared populations of nucleic acid molecules for sequencing. Examples of automated sample preparation systems are described in U.S. patent application Ser. No. 11/045,678, titled “Nucleic acid amplification with continuous flow emulsion”, filed Jan. 28, 2005, which is hereby incorporated by reference herein in its entirety for all purposes.

Also, the systems and methods of the presently described embodiments of the invention may include implementation of some design, analysis, or other operation using a computer readable medium stored for execution on a computer system. For example, several embodiments are described in detail below to process detected signals and/or analyze data generated using SBS systems and methods where the processing and analysis embodiments are implementable on computer systems.

An exemplary embodiment of a computer system for use with the presently described invention may include any type of computer platform such as a workstation, a personal computer, a server, or any other present or future computer. Computers typically include known components such as a processor, an operating system, system memory, memory storage devices, input-output controllers, input-output devices, and display devices. It will be understood by those of ordinary skill in the relevant art that there are many possible configurations and components of a computer and may also include cache memory, a data backup unit, and many other devices.

Display devices may include display devices that provide visual information, this information typically may be logically and/or physically organized as an array of pixels. An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art.

In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof.

A processor may include a commercially available processor such as a Centrino®, Core™ 2, Itanium® or Pentium® processor made by Intel Corporation, a SPARC® processor made by Sun Microsystems, an Athalon™ or Opteron™ processor made by AMD corporation, or it may be one of other processors that are or will become available. Some embodiments of a processor may include what is referred to as Multi-core processor and/or be enabled to employ parallel processing technology in a single or multi-core configuration. For example, a multi-core architecture typically comprises two or more processor “execution cores”. In the present example each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system, which may be, for example, a Windows®-type operating system (such as Windows® XP or Windows Vista®) from the Microsoft Corporation; the Mac OS X operating system from Apple Computer Corp. (such as Mac OS X v10.5 “Leopard” or “Snow Leopard” operating systems); a Unix® or Linux-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may include any of a variety of known or future memory storage devices. Examples include any commonly available random access memory (RAM), magnetic medium such as a resident hard disk or tape, an optical medium such as a read and write compact disc, or other memory storage device. Memory storage devices may include any of a variety of known or future devices, including a compact disk drive, a tape drive, a removable hard disk drive, USB or flash drive, or a diskette drive. Such types of memory storage devices typically read from, and/or write to, a program storage medium (not shown) such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modem cards, wireless cards, network interface cards, sound cards, or other types of controllers for any of a variety of known input devices. Output controllers could include controllers for any of a variety of known display devices for presenting information to a user, whether a human or a machine, whether local or remote. In the presently described embodiment, the functional elements of a computer communicate with each other via a system bus. Some embodiments of a computer may communicate with some functional elements using network or other types of remote communications.

As will be evident to those skilled in the relevant art, an instrument control and/or a data processing application, if implemented in software, may be loaded into and executed from system memory and/or a memory storage device. All or portions of the instrument control and/or data processing applications may also reside in a read-only memory or similar device of the memory storage device, such devices not requiring that the instrument control and/or data processing applications first be loaded through input-output controllers. It will be understood by those skilled in the relevant art that the instrument control and/or data processing applications, or portions of it, may be loaded by a processor in a known manner into system memory, or cache memory, or both, as advantageous for execution.

Also a computer may include one or more library files, experiment data files, and an internet client stored in system memory. For example, experiment data could include data related to one or more experiments or assays such as detected signal values, or other values associated with one or more SBS experiments or processes. Additionally, an internet client may include an application enabled to accesses a remote service on another computer using a network and may for instance comprise what are generally referred to as “Web Browsers”. In the present example some commonly employed web browsers include Microsoft® Internet Explorer 7 available from Microsoft Corporation, Mozilla Firefox® 2 from the Mozilla Corporation, Safari 1.2 from Apple Computer Corp., or other type of web browser currently known in the art or to be developed in the future. Also, in the same or other embodiments an internet client may include, or could be an element of, specialized software applications enabled to access remote information via a network such as a data processing application for SBS applications.

A network may include one or more of the many various types of networks well known to those of ordinary skill in the art. For example, a network may include a local or wide area network that employs what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

b. Embodiments of the Presently Described Invention

As described above, the presently described invention comprises methods for measurement of a level of activity associated with one or more enzyme reagents, and dynamically adjusting the concentration of one or more of the measured enzyme reagents to provide a substantially optimal level of activity of the reagents. In particular, the presently described invention may be employed to reduce introduced errors in sequence data attributable to carry forward and incomplete extension effects caused by undesirable levels of nucleotide species at certain process steps as well as modulating the degree of detectable signal output from a sequencing process.

Some embodiments of the presently described invention may be employed as a means for calibrating the signal output between multiple sequencing instruments and/or sequence runs. Also, excessively high or low signal variation may be reduced as well as “signal bleed” effects (i.e. a signal from one flow is so high that the signal persists and bleeds into the next flow, or in some cases into neighboring wells or reaction sites during the same or subsequent flow). In some embodiments, calibration and reduction of signal variations and/or bleed allows for shorter flow times resulting in more frequent and efficient cycling through flows (i.e. shorter flow cycles). For example, it is generally desirable that the signal output for each incorporation event is within an acceptable range so that the output is more comparable between instruments and sequencing runs as well as increasing the data quality and reliability.

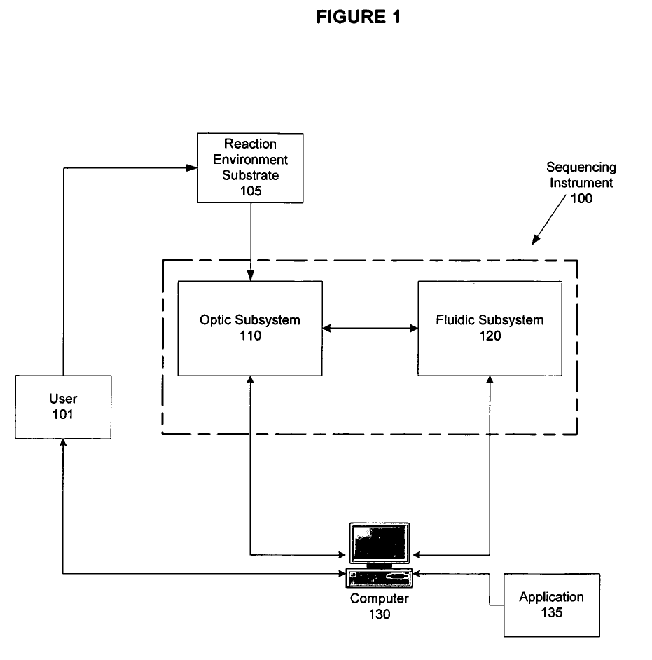

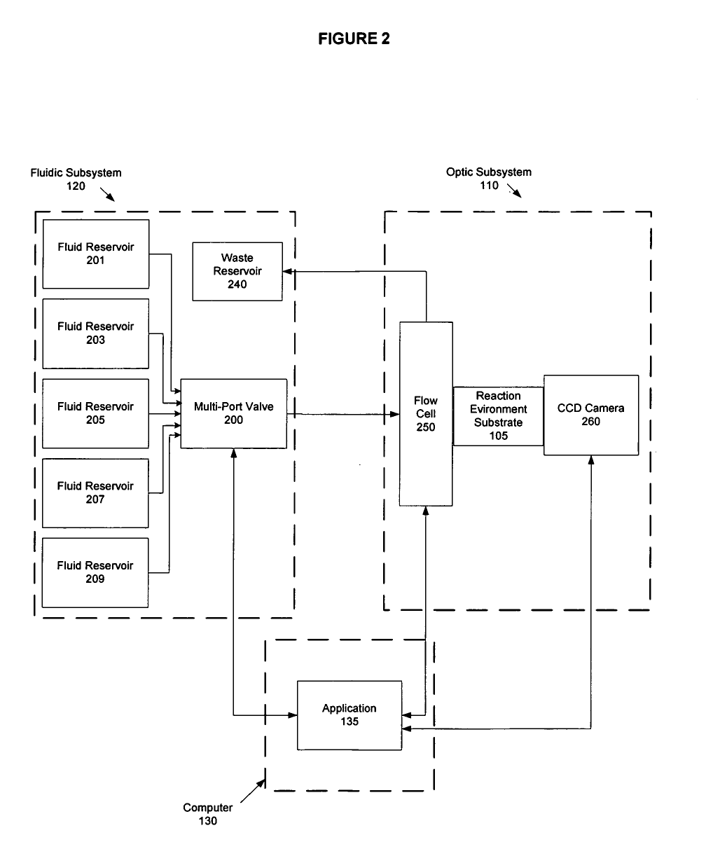

Typically, one or more instrument elements may be employed that automate one or more process steps for the measuring and adjusting. For example, embodiments of a sequencing method may be executed using instrumentation to automate and carry out some or all process steps. FIG. 1 provides an illustrative example of sequencing instrument 100 that comprises optic subsystem 110 and fluidic subsystem 120. Embodiments of sequencing instrument 100 employed to execute sequencing and adaptive control processes may include various fluidic components in fluidic subsystem 120, various optical components in optic subsystem 110, as well as additional components not illustrated in FIGS. 1 or 2 that may include microprocessor and/or microcontroller components for local control of some functions. Further, as illustrated in FIG. 1 sequencing instrument 100 may be operatively linked to one or more external computer components such as computer 130 that may for instance execute system software or firmware such as application 135 that may provide instructional control of one or more of the components and/or some data analysis functions. In the present example, sequencing instrument 100 and/or computer 130 may include some or all of the components and characteristics of the embodiments generally described above.

Embodiments of fluidic subsystem 120 may include various components such as fluid reservoir 201, fluid reservoir 203, fluid reservoir 205, fluid reservoir 207, and fluid reservoir 209 that each may hold a volume of reagent or fluid usable in a process step such as would occur with a sequencing reaction procedure. For example, reservoirs 201-209 may include bottles, flasks, tubes, cuvettes, or other fluid tight receptacle that hold volumes of reagents such as individual nucleotide species (i.e. A, C, G, T, or U); specific enzymes such as apyrase, sulfurylase, luciferase, PPi-ase or other enzyme; test or calibration fluids that may include ATP or PPi; substrates; enzyme enhancers or inhibitors; buffers or wash solutions that may include that may include counter ions (i.e. Ca²⁺ or Mg²⁺) and/ or preferred PH, water and/or dilutions of a bleach, iodine, or other disinfecting solution; or other fluid useable in the sequencing process or for preparation thereof. Embodiments of fluidic subsystem 120 may also include one or more waste reservoirs 240 for recapture and storage of used or spent fluids that are undesirable for re-use. It will be appreciated that multiple embodiments of reservoir 240 may be employed for fluids that are incompatible or dangerous to combine or generally preferable to keep separated.

Further each of reservoirs 201-209 may be in fluid communication with multi-port valve 200. Multi-port valves are generally known to those of skill in the art and are available from suppliers such as SMC Corporation of Indianapolis, Ind. In the example of FIG. 2, multi-port valve 200 is enabled to open and close to selectively allow specified volumes of fluid to move from reservoirs 201-209 to flow cell 250. The period of opening and closing may be referred to as “pulse width” and is generally measured as units of time that a valve spends open (i.e. may be measured in milliseconds or other similar measure). In some instances, the pulse width associated with the introduction of one fluid may occur simultaneously with the introduction of another fluid, where the two fluids may mix together. Further, multi-port valve 200 is enabled to adjust rates of flow from one or more of reservoirs 201-209 simultaneously. In the present example, the adjustable flow rate or pulse width timing permits for accurate control of concentration and dilution of reagents. For instance, one possible method for controlling a 10× dilution of a reagent may be achieved by opening a flow from one of reservoirs 201-209 with a pulse width equal to 1/10^{th} of the total time that all reagents associated with a process step are allowed to flow through flow cell 250. In some embodiments, valve 200 may be “pulsed” at intervals (i.e. open vs. closed intervals with shorter pulse widths) to provide better homogeneity of the dilution.

Some alternative embodiments may also include controlling a flow rate through valve 200 by modulating the degree that valve 200 is opened. In other words, valve 200 may be opened through a range of a small partial opening to completely open where the rate of flow is dependent upon the degree of opening. It will be appreciated that it may be desirable that the concentrations of one or more reagents in reservoirs 201-209 are exactly known. It may further be desirable that the concentrations of the reagents in reservoirs 201-209 are higher, and in some case substantially higher, than what is desirable for a sequencing process, thus allowing for dilution and easy management of final concentration.

Those of ordinary skill in the related art will also appreciate that fluidic subsystem 120 is exemplary and other components may be included in a typical fluidic subsystem. For instance, some embodiments may include sensors enabled to detect fluid volume in a reservoir and/or that the correct or expected fluid is present in reservoirs 201-209 or flowing through valve 200. Sensors may include a combination of one or more sensors such as conductivity sensors, optical sensors (i.e. optical density), acoustic sensors (i.e. ultrasonic density), or PH sensors. Some typical fluidic components in subsystem embodiments may also include valves, tubing, pumps (i.e. peristaltic pumps), heating/cooling elements (i.e. heat sink), or other elements commonly employed in the art.

Also illustrated in FIG. 2 are components associated with optic subsystem 110 that include flow cell 250, reaction substrate 105, and detector 260. For example, flow cell 250 is in fluid communication with a first surface of reaction environment substrate 105 that in some embodiments include the wells of a PTP plate housing many populations of substantially identical template nucleic acid molecules. Thus the fluid introduced into flow cell 250 is contacted with substrate 105 and the template nucleic acid molecules. Also, in some embodiments what is referred to as a “convective” flow may be established within flow cell 250 for efficient introduction and removal of the reagents from substrate 105. Examples of convective flow in sequencing embodiments and its advantages are described in U.S. patent application Ser. Nos. 10/191,438; 11/016,942; 11/217,194, each of which are hereby incorporated by reference herein in its entirety for all purposes. Additionally, as described above detector 260 may include an embodiment of CCD camera, Photo Multiplier Tube (PMT), or other optical detection device known to those of ordinary skill in the art. Preferably, detector 260 is in optical communication with a surface of reaction substrate 105 so that light generated from sequencing reactions is transmitted to detector 260. It will also be appreciated that in some embodiments flow cell 250 and reaction environment substrate 105 are the same element where flow cell 250 may include one or more interior surfaces that act as substrate 105.

As described above, embodiments of the presently described invention dynamically control the concentration of one or more enzyme reagents delivered to a reaction environment, which for instance may include control of concentration and delivery via multi-port valve 200 to reaction environment substrate 105. For example, computer 130 may include application 135 stored for execution in system memory that provides instructional control of the dynamic concentration adjustments. In some embodiments, application 135 also receives input for calculating concentration adjustments, determines the desired adjustment to implement, and provides instructional control to one or more elements of sequencing instrument 100 such as microcontroller elements for timing control of valve 200. In the present example, application 135 may initiate the sequencing process with initial concentration values for one or more enzyme reagents and adjust the concentrations at predefined time intervals (i.e. may employ an open-loop type mechanism for pre-defined dilutions profiles) or in response to a measured change where the concentration of the reagents may be continuously or periodically measured during a sequencing run (i.e. may employ a closed-loop type of feedback control mechanism for measured reagent activities).

Those of ordinary skill in the related art will appreciate that the effectiveness or activity of various embodiments of enzyme reagents may vary for a variety of reasons such as lot to lot variation, variation between manufacturers, degradation over time (i.e. shelf life), environmental effects (i.e. temperature, humidity, etc.), and other effects known or unknown. Thus, it is generally advantageous to adjust the concentration of an enzyme reagent in a reaction based, at least in part, upon its level of activity. Those of ordinary skill in the related art will appreciate that enzyme activity is generally measured in enzyme units (U) where one U is equal to the amount of enzyme that catalyzes the conversion of one micro mole of substrate in one minute. Another unit of measure sometimes employed for enzyme activity is referred to as a katal. It will also be appreciated that certain environmental conditions affect the catalytic rates of enzymes such as temperature and PH, and it is generally desirable to quantify and/or control such conditions when measuring and employing enzyme reagents in sequencing processes. Importantly, the term “concentration” of an enzyme reagent as used herein refers to the level of activity of the enzyme reagent per unit of volume (generally the units of measure may include as U/ml).

As generally described above, some enzymes diminish in levels of catalytic activity over time which in some cases results in a substantial loss of catalytic activity. The rate of loss of such activity depends on a number of factors that include storage conditions and type of enzyme, where it may be extremely difficult to predict the rate of change without direct measurement. Further, some sequencing process may require extended periods of time to execute a complete run where some reagents, such as for instance apyrase, may be especially sensitive to some environmental conditions that can be particularly problematic in combination with the extended time periods. In some embodiments, user 101 may not be aware of the loss of catalytic activity where the adaptive control described herein may avoid consequences that could include the catastrophic loss of an entire experiment.

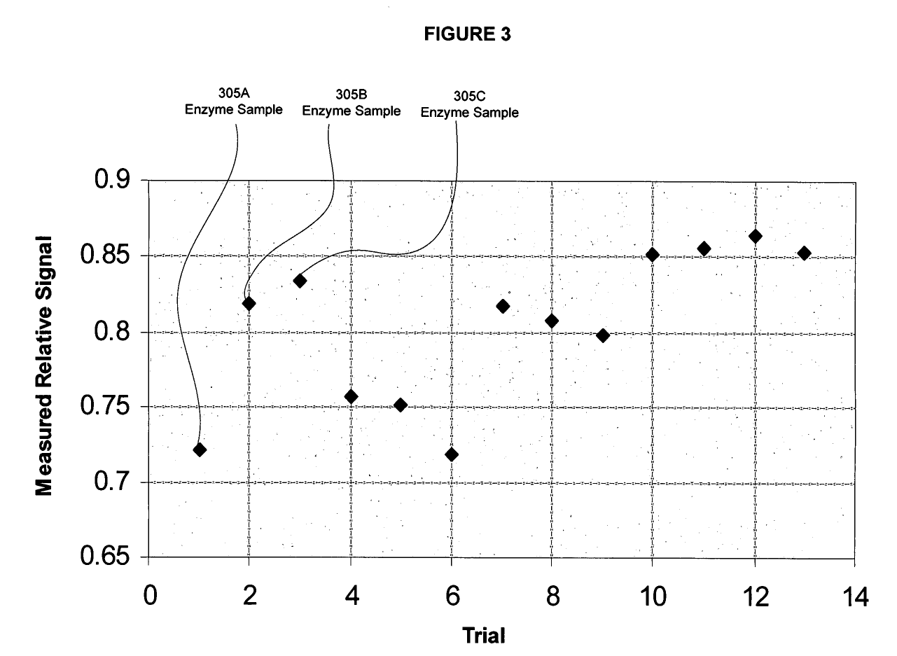

FIG. 3 provides an illustrative example of measured variation in the activity of different samples of apyrase enzyme having a presumed concentration that is the same for all samples. The pulse width (i.e. volume) was the same for each of the measured samples but each were measured as having substantially different levels of detected relative signal and thus activity. In the present example, the “Measured Relative Signal” for each of enzyme samples 305A, 305B, and 305C were measured using the amplitude measurement method described below and correlates with the level of activity of the enzyme in each sample. Importantly, it is clear that there is substantial variation in the level of activity among 13 different samples tested, and thus the effects of each enzyme sample on a sequencing process depends, in part, on the level of activity.

It will also be appreciated by those of ordinary skill that the measured activity of an enzyme reagent may depend, at least in part, upon the concentration of one or more other reagents that interact with the enzyme reagent in question that, for instance, may include substrates, enhancers, or inhibitors. For example, a desirable concentration of apyrase employed to inactivate a nucleotide specie depends in part upon the concentration of said nucleotide specie in the reaction environment of flow cell 250 and/or reaction substrate 105. In other words, if the concentration of nucleotide specie is high or low, the concentration associated with a desired activity of apyrase should be similarly high or low. Also, in some embodiments the reaction environment may include complexities that reduce diffusion or flow characteristics. In such cases, it may be desirable to adjust the apyrase concentration to account for such complexity.

As described above, embodiments of the presently described invention includes measuring the activity of one or more enzyme reagents as part of a sequencing process. For example, some embodiments of the invention measure the activity of one or more enzyme reagents employed in sequencing processes by running one or more test reactions using the enzyme reagents and measuring the result of the reaction to determine the level enzyme activity and adjust the enzyme concentration accordingly. Those of ordinary skill in the related art will appreciate that the methods for measuring enzyme activity depend, at least in part, upon the species of enzyme as well as other factors. Also, the measurement may be different for different enzyme reagents and in some cases a method of measurement may provide an indication of the activity of the enzyme reagent in the presence of a number of reagents that interact with the enzyme reagent. In some embodiments, it may be preferable to control enzyme reagent activity by modulating the concentration of one or more of the number of interacting reagents or environmental conditions. For example, the level of activity of apyrase may be adjusted using PH where a PH of about 6.5 is preferred for optimal apyrase activity. Thus, the activity of apyrase may be lowered by using a higher or lower PH, where the degree of difference relates to the degree of reduced activity. It will also be appreciated that it may be desirable to use a PH level in a sequencing process that is optimal for other enzymes or steps. In the present example, a PH of about 7.8 may be employed in a sequencing process that is optimized for performance of a polymerase enzyme. In such a case, it is generally preferable to measure and adjust apyrase activity using the preferred PH of the sequencing process.

Alternatively, in some embodiments of a sequencing process implemented by sequencing system 100 it is possible to modify environmental conditions to suit the optimal ranges of different enzymes. For example, due to the sequential nature of processing steps when apyrase is included in a wash step to remove excess nucleotide species and ATP, a buffer may be employed with the apyrase with a PH that optimizes the apyrase activity level. Subsequently, during the next nucleotide incorporation step using a polymerase enzyme a different buffer with optimal PH conditions for the polymerase may be employed in order to optimize the polymerase activity. In addition, each optimized buffer could include preferred counter ions for each enzyme such as Ca²⁺ for the apyrase buffer and Mg²⁺ for the polymerase buffer.

One embodiment of measurement technique for apyrase may be referred to as “Phase Measurement” that employs a special test molecule designed to exaggerate errors introduced by sub-optimal apyrase concentration. In particular, the carry forward effect of low apyrase concentrations described above is easily measured by sequencing instrument 100 and is further easily correlated with the relative catalytic activity of the enzyme. For example, a test molecule may include a sequence composition of nucleotide species that include: GCGCCCCCCCC (SEQ ID NO 1). Importantly, the test molecule comprises a sequence composition that produces a measurable error in a minimum number of flow cycles. In the present example, because the exact sequence of the test molecule is known and comprises only the C and G nucleotide species, a special sequencing protocol may be employed that only introduces flows of the C and G nucleotide species. The use of just 2 nucleotide species conserves on reagent usage as well as avoids reactions with sample molecules that begin with A or T nucleotide species that may be present in the reaction environment. For instance, an adaptor element associated with every substantially identical template molecule may include an A or T nucleotide specie in the first sequence position, or in some embodiments the first several sequence positions (i.e. may include a range of 2-10 sequence positions). Thus, using a test molecule with G and C nucleotide specie composition may be employed in the presence of the template molecules without corrupting the sequence data. Further, the concentration of apyrase may be modified in the test protocol to produce exaggerated effects that provide a baseline to incrementally increase or decrease concentration as needed. For instance, the apyrase concentration employed using a pulse width that may be 1/10^{th} the “normal” or optimal concentration typically employed in a sequencing process to produce introduced carry forward error in the resulting sequence data. Importantly, even though the concentration of apyrase may be lower than a standard concentration it is relatively easy to determine the catalytic activity of the apyrase in the system and to calculate the dilutions of the apyrase to achieve a desired concentration for use in a sequencing run.

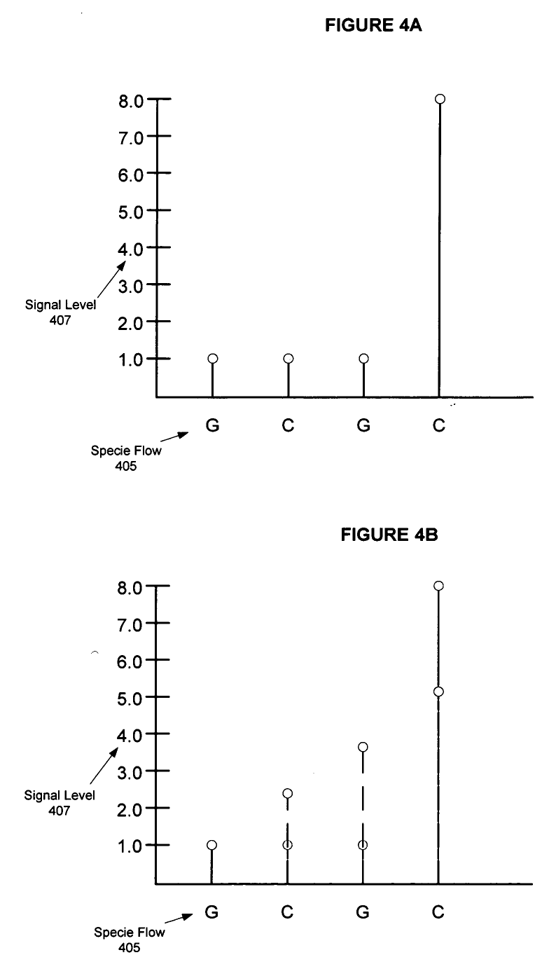

A exemplary flowgram without any introduced carry forward error that would be expected to be generated from the test molecule of the present example is illustrated in FIG. 4A where signal level 407 correlates exactly with the sequence composition of the test molecule in specie flow 405. In other words, a value of signal level 407 of 1.0 is representative of the sequence composition of the first three sequence positions that comprise a single nucleotide specie in the test molecule. A value of 8.0 is representative of the run of 8 homopolymers of the next sequence positions in the test molecule.

Continuing with the example from above, using low apyrase concentration and assuming that simple washing of the reaction substrate is not sufficient to remove all nucleotide species from previous runs, the sequence data generated from the test molecule produces a measurable carry forward error. FIG. 4B illustrates an example of the possible effects of carry forward error for species flow 405 on the test molecule. The heavy dashed lines represent the signal level 407 with carry forward error for each specie flow 405. For instance, with low apyrase concentration there is residual G nucleotide specie leftover from the 1^{st} flow present in the reaction environment during the 2^{nd} flow of the C nucleotide specie resulting in incorporation of both nucleotide species (i.e. incorporation at the 2^{nd} C and 3^{rd} G sequence positions of the test molecule, and possibly at the 4^{th}-8^{th} C positions) that produces light from both incorporated species and a measurable increase in detected signal level. The measurable increase is indicative of the amount of residual G nucleotide specie present during the 2^{nd} flow and correlates with the level of activity of the apyrase (i.e. the low concentration that did not degrade all of the G nucleotide molecules). The effect is further exaggerated on the 3^{rd} flow, where there is residual C nucleotide specie from the 2^{nd} flow in the reaction environment during the 3^{rd} flow and a similarly measurable carry forward effect results. Finally, a 4^{th} flow of the C specie may be employed if necessary and illustrates a measurably reduced signal from the carry forward effects representing the pre-extension of the C nucleotide species from the previous flows. In other words, because of the carry forward extensions in the previous flow cycles there are fewer test molecules in phase (or phasic synchrony) with the flow of the C nucleotide specie during the 4^{th} flow, and thus fewer incorporation events that results in a lower detected signal than what would be expected if carry forward error was not present. It will be appreciated that in the present example the detected signal value is still brighter than the previous flows which may be useful for identification of wells comprising the test molecules.

Another technique for measuring apyrase activity is referred to herein as “Amplitude Measurement”. Amplitude measurement has some advantages over the phase measurement method because no special test molecules are required, where instead amplitude measurement may employ ATP as a reaction substrate that will not affect other steps in the sequencing process. Further, the amplitude measurement technique employs simple flow algorithms and signal processing methods (does not require application 135 to implement a well finding algorithm). In addition, amplitude measurement may be more amenable to frequent measurement without introducing the risk of phase error (i.e. by adding dNTP species during the measurement that could incorporate into nascent strands associated with template nucleic acid molecules), and is less sensitive to other sources of background error.

As mentioned above, some embodiments of amplitude measurement employs a characteristic of apyrase that converts an Adenosine Triphosphate substrate (generally referred to as ATP) to Adenosine Diphosphate (generally referred to as ADP) rendering it functionally inactive in pyrophosphate sequencing processes. ATP is generally appreciated as important in cellular metabolism for energy transfer and a fundamental reaction substrate for catalysis of luciferin by the luciferase enzyme producing light as one reaction product. It will also be appreciated that luciferin and luciferase are typically used in pyrosequencing methods that ultimately results in the generation of light when a nucleotide specie is incorporated. Thus, the efficiency of apyrase activity on its ability to hydrolyze/degrade ATP may be easily measured in the presence of luciferin and luciferase by measuring the light output using the light detection capabilities of system 100. Other reaction substrates may also be employed as an alternative to ATP in some embodiments that include other nucleotide triphosphates (i.e. CTP, GTP, or TTP), ribonucleotides, or deoxynucleotides (such as deoxynucleotide triphosphates, dNTP's). It will however be appreciated that there is a risk of the incorporation of dNTP's by polymerase into a nascent strand as described above with respect to the phase measurement technique and thus may not be ideal for some applications.

In some embodiments of sequencing processes employing amplitude measurement it may be advantageous to determine the apyrase activity for each dNTP species employed in the sequencing reactions. As mentioned elsewhere in the present description apyrase may be employed to degrade both ATP and dNTP species during a wash phase where the apyrase activity may vary for the different dNTP species. For example, a measurement technique may be employed that separately tests each dNTP species in an excess concentration with ATP present in a limiting concentration. Thus, the dNTP acts as a competitor for the ATP with the apyrase and the efficiency of the apyrase to degrade each nucleotide species can be determined. Then, the optimal concentration of apyrase for each nucleotide species may be employed after each flow with the respective nucleotide species.

A first embodiment of amplitude measurement includes concurrently introducing a concentration of apyrase and a concentration of an ATP reaction substrate into a reaction environment, measuring the output of light as a reaction product, and correlating the measured output with a level of enzyme activity. In the described embodiment, the apyrase activity is not dependent upon the concentration of ATP substrate in the reaction environment where the reaction is in “first order” for the ATP. However, it is still generally advantageous to use a known ATP concentration. In some embodiments of instrument 100, luciferin, luciferase, and sulfurylase enzymes are present in the reaction environment in concentrations in excess than what is called for by the sequencing process alone where the excess concentration has no deleterious effect on other interactions or processes. In fact, the excess concentration of luciferin, luciferase, and sulfurylase may be useful to counteract possible low catalytic activity effects of the luciferase or sulfurylase enzymes (methods of the present invention may also be employed to measure luciferase and/or sulfurylase activity as will be described in greater detail below). Thus, when an ATP reaction substrate is introduced into the reaction environment in the absence of inhibitory effects it would react with the luciferin and luciferase and result in light production. At the same time the apyrase acts to inhibit the light producing effects by converting the ATP to ADP. Thus the amount of light produced from the addition of ATP is indicative of the apyrase activity and efficiency of converting the ATP to ADP. For example, multi-port valve 200 may measure and introduce desired concentrations of the apyrase and ATP into flow cell 250. In the present example, the apyrase and ATP reagents may be introduced into flow cell 250 in parallel as a combined fluid or serially as distinct fluids (i.e. in sequence where the apyrase is added first and the ATP is added second or vice versa). Continuing with the present example, the apyrase may act immediately to convert the ATP to ADP upon introduction into flow cell 250 and a measurable rate of activity may be derived from light generated when the ATP comes into contact with the luciferase enzymes.

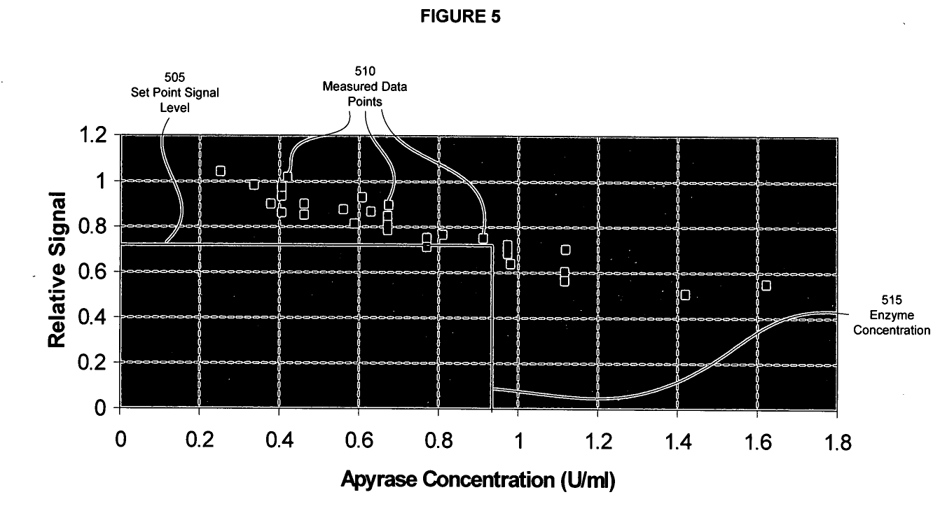

FIG. 5 provides an illustrative example of the relationship between the relative signals detected from the input of different apyrase concentrations with an ATP reaction substrate into the reaction environment. Also illustrated in FIG. 5 are set point signal level 505 and enzyme concentration 515 that indicate the desired levels for use in sequencing processes. Importantly, the relationship between the detected relative signals and the apyrase concentration is linear, thereby enabling easy determination of adjustments to a stock apyrase solution to achieve the concentration with desired activity level. For example, a regression line may be drawn through measured data points 510 to illustrate the linear relationship. Also, in the present example the optimal concentration for enzyme concentration 515 includes a value of 0.95 U/ml.

A second embodiment of amplitude measurement may include concurrently or sequentially introducing a concentration of apyrase and a concentration of a PPi reaction substrate into the reaction environment. Sulfurylase catalyzes the PPi producing ATP as a reaction product, and thus the activity of the apyrase enzyme may be measured as described above. One possible drawback of using PPi as a test or reaction substrate would be experienced in embodiments of instrument 100 and methods that employ a PPi-ase enzyme to degrade excess PPi (i.e. may be employed in a flow and/or immobilized upon solid phase bead substrates in the reaction environments). Therefore, PPi-ase activity may disrupt measurement of apyrase activity. However, measurements of PPi-ase activity may be made using the presently described embodiment (i.e. by flowing PPi with PPi-ase as competitors in the absence of apyrase for a period to establish a baseline estimation of PPi and PPi-ase activity). Further, the ATP generated by the PPi flow may be regionally specific since it will present at the site of production and not as ubiquitously distributed as when introduced through valve 200. Examples of embodiments that employ PPi-ase in sequencing processes are described in U.S. Provisional Patent Application Ser. No. 61/026,547, titled “System and Method for Improved Signal Detection in Nucleic Acid Sequencing”, filed Feb. 6, 2008, which is hereby incorporated by reference herein in its entirety for all purposes.

It will also be appreciated that embodiments for measurement and adaptive control of concentration of other enzyme reagents such as luciferase and sulfurylase may also be performed using competitive reaction substrates. For example, variation in signal output may be due, at least in part, to the activity or concentration levels (may also include spatial effects where localized regions may have higher or lower concentrations) of luciferin, luciferase, or sulfurylase as well as the concentration and distribution of template molecules. In the present example, some embodiments of sequencing instrument 100 may employ luciferase and sulfurylase enzymes bound to beads that are disposed in reaction wells with a population of substantially identical template molecules which may also be disposed upon one or more beads. The arrangement of beads may result in what are referred to as “layering effects”. The term layering effects as used herein generally refer to differences in localized distribution of substrates, reagents, targets, etc. that may be the result of processes used to distribute the subject material in the reaction environment. The result may include localized variance in the concentration of one or more of the enzymes relative to each other or to populations of template molecules or some combination thereof.

In addition to modification of pulse width for a particular enzyme reagent, one embodiment for control of system gain and calibration includes employing what are known as “inhibitors” that modulate the level of activity of an enzyme. The term “inhibitor” as used herein generally refers to a relationship between interacting molecules where one molecule, the inhibitor, exerts a negative influence in the activity of the other. In the presently described invention, it may be desirable to modulate the activity of the sulfurylase and luciferase enzymes that are important in a cascade of reactions that result in light generation in response to the incorporation of a nucleotide specie and release of PPi. For example, an inhibitor of the luciferase enzyme such as CAPMBT may be introduced into the reaction environment of flow cell 250 and reaction substrate 105 via multi-port valve 200. In some embodiments the inhibitor is added under the control of application 135 in response to a measured light output from one or more incorporation events. The light output may be measured in a number of ways, such as measuring output from a sequence having a known composition that may include a key sequence or the test molecule described above. Further, the light output may be measured from flows of ATP or PPi as described above which may also be useful for measuring what may be referred to as “signal decay” or “signal droop” that may occur during a sequencing run. Multi-port valve 200 under control of application 135 may add the appropriate concentration of inhibitor to reach a desired level of enzyme activity. In some embodiments the process may also be iterative, where subsequent rounds of measurement and calibration may be performed until a desired result is achieved.

Another embodiment for control of system gain and calibration includes modulating one or more “substrate” reagents, where an increase in the amount or concentration of available substrate results in an increase in signal output. For example, the light output from flows of ATP or PPi as described above may be employed to modulate activity associated with substrates that include D-luciferin and/or APS. As described above for system gain control using inhibitors, multi-port valve 200 under control of application 135 may add the appropriate concentration of substrate to reach a desired activity level. Yet another embodiment may include modulating some or all of the enzymes, inhibitors, or substrates described above in some combination.

A further embodiment may include modulation of activity level of an enzyme using environmental conditions such as PH. For a number of enzymes, the level of an enzyme activity may depend, at least in part, upon the relationship of the PH level in the reaction environment as compared to the optimal PH level for the activity of an enzyme. It is important to note that activity levels of different enzymes may typically vary from each other with respect to their optimal conditions, and that it may be advantageous to employ one or more enzymes in conditions that may be sub-optimal but may be compensated for using higher concentrations or volumes. For example, an optimal PH for apyrase activity may include a value of 6.5, where an optimal PH for polymerase may include a value of 7.8.

As described above, elements of the presently described invention are directed to the adaptive control of one or more enzyme reagents in nucleic acid sequencing processes. Generally, embodiments of the invention include measuring the activity of one or more enzyme reagents prior to or simultaneous with the initiation of a sequencing run, at regular intervals during a sequencing run, consistent monitoring during a sequencing run, or some combination thereof. In some embodiments it may be highly desirable to conduct multiple measurements to enable measurement of statistical significance and control for error in the measuring and/or calibration process as well as temporal changes to conditions in the system. Taking multiple measurements is useful to determine a measure of statistical significance to arrive at a more accurate adjusted final concentration of an enzyme reagent. For example, the measurement process may include a series of flows of apyrase with an ATP reaction substrate at three different apyrase volumes (determined by pulse width) and the signal output of light measured for each flow. The concentration of ATP does not need to be precise, but could include a concentration of about 0.875 μM. In the present example, the series includes 3 flows of the apyrase at each of the 3 pulse widths that include 83 ms (expected concentration of about 0.0079 U/ml), 138 ms (expected concentration of about 0.0131 U/ml), and 201 ms (expected concentration of about 0.019 U/ml). The measured relative signal from the flows at each pulse width are averaged and a regression line plotted through the averaged points and employed to determine a correction factor. After adjustment of the apyrase concentration, the process may be repeated with the adjusted concentration to verify that the adjustment is accurate.

As described, subsequent to a measuring step the activity of one or more enzyme reagents is modulated using concentrations or dilutions of the reagents, or enhancers, inhibitors, or substrates that interact with said reagents. The modulation may be implemented using “open loop” or “closed loop” strategies modulating the concentration of the enzyme reagents in a reaction environment or via manipulation of one or more parameters or conditions within the reaction environment (i.e. temperature, flow rate, PH, etc.). The term “open loop” as used herein generally refers to a fixed predetermined setting that is not responsive to feedback. The term “closed loop” as used herein generally refers to a system of feedback control where the amount of modulation is modified in response to measured parameters. The strategies of open and closed loop modulation may each provide advantages in various embodiments. For example, embodiments that fall into two general categories include what may be referred to as Signal Decay Compensation and Apyrase Compensation.

For example, advantages to employing an open loop modulation strategy in embodiments that apply correction or compensation for signal decay includes increasing the signal output capacity by enhancing light generation through reduction in inhibitor concentration and/or increasing substrate concentration as described above. Further, advantages to employing an open loop modulation strategy in embodiments that apply correction or compensation for apyrase activity includes an increase in concentration to counter for degradation of catalytic activity, or accumulation of phasic synchrony error. Also, the advantage includes a decrease in concentration to counter for a loss of polymerase or polymerase activity. In the present example, the open loop strategy is advantageous because both embodiments comprise a level of stability and/or predictability where significant or unpredictable changes are unlikely to occur within the duration of a sequencing run. Further, the advantage extends to data quality and comparability, where it is desirable for data consistency during a sequencing run.

Continuing with the present example, advantages to employing a closed loop modulation strategy in embodiments that apply correction or compensation for signal decay and for apyrase includes finer adjustments to produce higher quality and more reliable data. Also, a closed loop modulation strategy may be preferable in embodiments where the level of predictability and/or stability is low relative to a desirable threshold.

Also as described above, the level catalytic activity of one or more enzymes may depend, at least in part, upon conditions within a reaction environment. For example, the rate of degradation of apyrase may be temperature dependent, increasing in the rate of degradation as the temperature increases. The result may include an unpredicted drop in apyrase concentration leading to increased carry forward error. Alternatively, cooler temperatures may slow the rate of degradation and allow excessive concentrations of apyrase to build up leading to increased incomplete extension error. In the present example, the temperature may be measured in instrument 100 and modulated under the control of application 135 to maintain performance within desired parameters. The temperature may also be employed to apply modulation to apyrase activity to account for other activity effects as described above.

### EXAMPLE 1

### Verification of Adaptive Apyrase Process

Verification of the process for measurement of apyrase activity, determination of the pulse width correction factor required to achieve a predetermined apyrase activity, and adjustment of the apyrase pulse by the correction factor.

Apyrase activity means: Amount of ATP that apyrase can degrade as measured by the enzymatic activity in the wells;

Correction factor means: Multiplicative factor of pulse width required to obtain a predetermined apyrase activity;

Relative signal means: The measure of apyrase activity. The signal generated by a pulse of ATP with a concentration of apyrase normalized by the signal generated by the ATP only; and

Set point means: The predetermined relative signal desired. This is the relative signal the correction factor should correct the apyrase pulse width to measure.

A script was written which performs the apyrase activity measurement flows. The calculation of the correction factor was performed and then applied to the nominal apyrase activity measurement flow to determine the accuracy of the correction factor. In a standard script the correction factor was applied to the apyrase pulse in the washing kernel. The script was referred to as the ‘measure-adjust-remeasure’ script. It performed the apyrase measurement flows, adjustment of the apyrase pulse width and then remeasured the apyrase activity three times at the adjusted pulse. The average of these three measurements were taken as the metric to determine the accuracy of the adjustment factor.

The following tests were performed:

| Test | | | |
|---|---|---|---|
| No. | Description | Specification/Test | Result (mean ± SD) |
| 1 | Test on FLX293 | Relative error in the relative signal | 2.17% ± 2.98% |
| | with standard | generated by the corrected apyrase | 1.48% ± 0.68% with |
| | apyrase | pulse width and the set point 0.79 | one remeasure flow |
| | concentration | | outlier removed |
| 2 | Test on FLX312 | Relative error in the relative signal | 1.57% ± 0.3% |
| | with standard | generated by the corrected apyrase | |
| | apyrase | pulse width and the set point 0.79 | |
| | concentration | | |
| 3 | Test on FLX284 | Relative error in the relative signal | 2.66% ± 1.22% |
| | with 10% | generated by the corrected apyrase | |
| | increased in | pulse width and the set point 0.79 | |
| | apyrase | | |
| | concentration | | |
| 4 | Test on Rig 1 | Relative error in the relative signal | 2.15% ± 1.80% |
| | with 10% | generated by the corrected apyrase | 1.83% ± 1.18% with one |
| | decreased apyrase | pulse width and the set point 0.79 | remeasure flow |
| | concentration | | outlier removed |

Note that the typical conversion from apyrase pulse to relative signal is ˜0.002 cnt/ms. Using the 138 ms measurement pulse as our nominal concentration, the conversion from x % relative error in the remeasured value to relative apyrase concentration error is (x/100*0.79)/(0.002*138) for a set point of 0.79. For example, a relative error in the relative signal of 1.48% (test 1) implied an error in apyrase concentration of 4.24%.

The results for Test 1 are illustrated below. The process verified the algorithms ability to determine the region of interest from the ATP only image, which are the regions used to calculate the apyrase measurement and correction factor.

The algorithm determines the center and width of each loaded lane and selects interior regions of interest. The ‘measure-adjust-remeasure’ cycle was performed six times and each of the six remeasure kernel includes three iterations. The average of these three remeasure steps were taken as the metric of interest.

The aaLog.txt is the summary of the adaptive apyrase results generated by the instrument and included in a run directory. This log summarizes the information shown above and the data for the first ‘measure-adjust-remeasure’ cycle and is annotated for clarity.

| | |
|---|---|
| nPixelsUnderDCOffset = 3988 (0.023770%) | Determines whether the image needs to be DC offset |
| adjusting dc offset | |
| found 2 regions | |
| region 0: center = 1511, width = 798 | |
| region 1: center = 2548, width = 820 | Results of the region of interest finding |
| range 0: start = 1245, end = 1777 | |
| range 1: start = 2282, end = 2814 | |
| process image block . . . | |
| found 1 cal points | |
| 3 samples at 172.755524, average = 0.836458 | 3 measurements are taken before any adapting is done |
| not enough cal points (1) | |
| process image block . . . | |
| found 3 cal points | |
| 4 samples at 82.644630, average = 0.915227 | The results of the first cycle of apyrase measurements |
| 4 samples at 137.931030, average = 0.786803 | |
| 4 samples at 200.000000, average = 0.688323 | |
| found 3 cal points | |
| 4 samples at 82.644630, average = 0.915227 | |
| 4 samples at 137.931030, average = 0.786803 | |
| 4 samples at 200.000000, average = 0.688323 | |
| 0: x = 82.644630, y = 0.936956 | |
| 1: x = 137.931030, y = 0.804354 | |
| 2: x = 200.000000, y = 0.690772 | |
| 3: x = 82.644630, y = 0.942456 | |
| 4: x = 137.931030, y = 0.807885 | |
| 5: x = 200.000000, y = 0.701268 | |
| 6: x = 82.644630, y = 0.915674 | |
| 7: x = 137.931030, y = 0.772797 | |
| 8: x = 200.000000, y = 0.675789 | |
| 9: x = 82.644630, y = 0.865824 | |
| 10: x = 137.931030, y = 0.762176 | |
| 11: x = 200.000000, y = 0.685462 | |
| y = −0.001926431 * x + 1.066854 (raw) | |
| found 3 cal points | |
| 4 samples at 82.644630, average = 0.915227 | |
| 4 samples at 137.931030, average = 0.786803 | |
| 4 samples at 200.000000, average = 0.688323 | |
| calPoint 82.644630 has 48 points | |
| initial cv = 0.035133 | |
| cv trimmed 48 original values to 48 | Outlying data is rejected. This could be due to a |
| sd trimmed 48 original values to 47 | flow anomaly, bubble, etc |
| calPoint 137.931030 has 48 points | |
| initial cv = 0.037847 | |
| cv trimmed 48 original values to 48 | |
| sd trimmed 48 original values to 45 | |
| calPoint 200.000000 has 48 points | |
| initial cv = 0.020899 | |
| cv trimmed 48 original values to 48 | |
| sd trimmed 48 original values to 46 | |
| y = −0.001951383 * x + 1.071890 (trimmed) | The regression line is calculated for the apyrase |
| found 3 cal points | measurement |
| 47 samples at 82.644630, average = 0.916581, lineFit = 0.910619 | |
| 45 samples at 137.931030, average = 0.790962, lineFit = 0.802734 | |
| 46 samples at 200.000000, average = 0.687038, lineFit = 0.681614 | |
| calSetPoint = 0.790000 | |
| pulseWidthAtSetpoint = 144.456635 | |
| adjust = 1.046787 | The correction factor is calculated |
| maximumPulseWidth = 151 | |
| nominalPulseWidth = 116.119 | |
| minimumPulseWidth = 60 | |
| e2motf = −22.000000 | |
| newApyraseWashPulseWidth = 122.581 | The new remeasure pulse width is calculated |
| adjusted pulse width is 123 | |
| changing micro's pulse width to 123 | The pulse width is changed on the micro (to the ms) |
| MsgType = 108, Index = 21, Pulse = 123 | |
| SCRIPT_PULSE_WIDTH_NOTIFY msg received | |
| MsgType = 204, Index = 21, Pulse = 123 | |
| process image block . . . | |
| found 1 cal points | |
| 3 samples at 172.755524, average = 0.829998 | The average of the 3 remeasure flows |

Over the 4 trials (24 total ‘remeasures’ values), at 90% confidence the algorithm corrected the pulse width to generate a relative signal within 5% of the set point at least 78% of the time (22 successes). If the two outliers are removed, it will be within 5% at least 89% of the time (24 successes). It was also verified that the microcontroller can control the apyrase pulse width to within 0.5 ms of the calculated pulse width.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

What is claimed is:

1. A method for adaptive reagent control, comprising:
a) introducing a first concentration of an enzyme reagent into a reaction environment with a reaction substrate, wherein the enzyme reagent and reaction substrate are constituent parts of a sequencing process;
b) measuring a level of activity of the first concentration of the enzyme reagent in the reaction environment, wherein the level of activity comprises a measurable product of a reaction between the enzyme reagent and the reaction substrate;
c) identifying an optimal concentration using the measured level of activity of the first concentration; and
d) performing the sequencing process in the reaction environment using the optimal concentration of the enzyme reagent, wherein the sequencing process comprises an iterative series of sequencing reactions.

2. The method of claim 1, further comprising:
before step d), repeating steps a) and b) using the optimal concentration as the first concentration; and
verifying the optimal concentration of the enzyme reagent using the measured level of activity.

3. The method of claim 1, further comprising:
introducing a second concentration and a third concentration of the enzyme reagent into the reaction environment with the reaction substrate;
measuring a level of activity of the second and third concentrations of the enzyme reagent in the reaction environment; and
identifying the optimal concentration using the measured level of activity of the first, second and third concentrations.

4. The method of claim 3, wherein:
measuring the level of activity for each of the first, second, and third concentrations two or more times, wherein the optimal concentration is identified using an average of the two or more measured levels of activity at each of the first, second and third concentrations.

5. The method of claim 1, further comprising:
e) repeating steps a)-c) one or more times during the sequencing process.

6. The method of claim 1, wherein:
the enzyme reagent comprises apyrase and the reaction substrate comprises ATP, wherein the ATP is introduced into the reaction environment with the apyrase.

7. The method of claim 6, wherein:
the iterative series of sequencing reactions are performed with a template nucleic acid, and the optimal concentration of the apyrase is introduced into the reaction environment prior to a subsequent iteration of sequencing reaction to reduce an introduced error in sequence composition of the template nucleic acid generated in the subsequent iteration.

8. The method of claim 7, wherein:
the introduced error comprises a carry forward error when a concentration of the apyrase in the reaction environment is lower than the optimal concentration.

9. The method of claim 7, wherein:
the introduced error comprises an incomplete extension error when a concentration of the apyrase in the reaction environment is higher than the optimal concentration.

10. The method of claim 7, wherein:
the introduced error comprises a low reaction product from the sequencing reaction when a concentration of the apyrase in the reaction environment is higher than the optimal concentration.

11. The method of claim 1, wherein:
the enzyme reagent comprises PPi-ase and the reaction substrate comprises PPi, wherein the PPi is introduced into the reaction environment with the PPi-ase.

12. The method of claim 1, wherein:
the enzyme reagent comprises apyrase and the reaction substrate comprises PPi, wherein the PPi is introduced into the reaction environment with the apyrase.

13. The method of claim 1, wherein:
the enzyme reagent is sensitive to an environmental condition, wherein the environmental condition changes the level of activity of the enzyme reagent.

14. The method of claim 13, wherein:
the environmental condition comprises PH or temperature.

15. The method of claim 1, wherein:
the measurable product comprises light emitted from the reaction.

16. The method of claim 1, wherein:
the sequencing reactions produce a plurality of reaction products that comprise the reaction substrate.

17. The method of claim 1, wherein:
the enzyme reagent degrades the reaction substrate.

18. The method of claim 1, wherein:
the reaction environment comprises a flow cell.

19. The method of claim 1, wherein:
the reaction environment comprises a well of a plate.

20. The method of claim 19, wherein:
the plate comprises a fiber optic faceplate comprising an array of the wells.

21. A nucleic acid sequencing system, comprising:
a flow cell that comprises a reaction environment for performing a sequencing process comprising an iterative series of sequencing reactions;
a valve that introduces a first concentration of an enzyme reagent into a reaction environment with a reaction substrate, wherein the enzyme reagent and reaction substrate are constituent parts of the sequencing process;
a detector that measures a level of activity of the first concentration of the enzyme reagent in the reaction environment, wherein the level of activity comprises a measurable product of a reaction between the enzyme reagent and the reaction substrate;
wherein in response to the measured level of activity the valve provides an optimal concentration of the enzyme reagent into the reaction environment.

22. The system of claim 21, further comprising:
a computer having executable code stored thereon, wherein the executable code performs the steps of:
providing instructional control for the valve to introduce the first concentration of the enzyme reagent and the reaction substrate into the reaction environment;
receiving the measured level of activity of the first concentration from the detector;
identifying an optimal concentration using the measured level of activity of the first concentration; and
providing instructional control for the valve to provide the optimal concentration.

23. The system of claim 22, wherein:
the instructional control is provided to a microcontroller that controls timing functions of the valve.

24. The system of claim 23, wherein:
the timing functions of the valve include control of a pulse width.

25. The system of claim 21, wherein:
the valve is a multiport valve.

26. The system of claim 21, further comprising:
a first fluid reservoir comprising a stock solution of the enzyme reagent and a second fluid reservoir comprising a stock solution of the reaction substrate, wherein the valve introduces the first concentration of the enzyme reagent and the reaction substrate into the reaction environment via the first and second fluid reservoirs.

27. The system of claim 26, wherein:
the valve introduces the optimal concentration via the first fluid reservoir.

28. The system of claim 21, wherein:
the detector is a CCD detector.

29. The system of claim 21, wherein:
the enzyme reagent comprises apyrase and the reaction substrate comprises ATP, wherein the ATP is introduced into the reaction environment with the apyrase.

30. The system of claim 29, wherein:
the valve provides the optimal concentration of the apyrase in each subsequent iteration of sequencing reaction after a first iteration to reduce an introduced error in a sequence composition generated in the subsequent iterations

31. The system of claim 30, wherein:
the introduced error comprises a carry forward error when a concentration of the apyrase in the reaction environment is lower than the optimal concentration.

32. The system of claim 30, wherein:
the introduced error comprises an incomplete extension error when a concentration of the apyrase in the reaction environment is higher than the optimal concentration.

33. The system of claim 30, wherein:
the introduced error comprises a low reaction product from the sequencing reaction when a concentration of the apyrase in the reaction environment is higher than the optimal concentration.

34. The system of claim 21, wherein:
the enzyme reagent is sensitive to an environmental condition, wherein the environmental condition changes the level of activity of the enzyme reagent.

35. The system of claim 34, wherein:
the environmental condition comprises PH or temperature.

36. The system of claim 21, wherein:
the measurable product comprises light emitted from the reaction.

37. The system of claim 21, wherein:
the sequencing reactions produce a plurality of reaction products that comprise the reaction substrate.

38. The system of claim 21, wherein:
the enzyme reagent degrades the reaction substrate.

39. The system of claim 21, wherein:
the reaction environment comprises a well of a plate.

40. The system of claim 39, wherein:
the plate comprises a fiber optic faceplate comprising an array of the wells.


SEQUENCE LISTING (1 sequence)

SEQ ID NO: 1 - DNA, 11 bp, Artificial Sequence
  Description of Artificial Sequence Synthetic oligonucleotide
gcgcccccccc